(19) United States
(12) Patent Application Publication
(10) Pub. No.: US 2014/0094762 A1
(43) Pub. Date: Apr. 3, 2014

(54) STABILIZED ADHESIVES AND USE THEREOF

(71) Applicants: Italo Corzani, Chieti (IT); Pietro Lunetto, Manoppello (IT)

(72) Inventors: Italo Corzani, Chieti (IT); Pietro Lunetto, Manoppello (IT)

(21) Appl. No.: 13/629,722

(22) Filed: Sep. 28, 2012

(51) Int. Cl. A61F 13/49; B32B 7/12; B32B 1/02; C09J 167/08

(52) U.S. Cl. CPC A61F 13/49; C09J 167/08; B32B 7/12; B32B 1/02
USPC 604/365; 524/386; 524/320; 524/239; 524/384; 156/327; 428/480; 428/35.7

(57) ABSTRACT

Disclosed herein are novel stabilizers for thermoplastic oil-modified alkyd resins. It has now been found that such stabilizers can greatly increase the stability of alkyd resins against melt viscosity variations after thermal aging. The alkyd resins can be used as the predominant component of a hot melt adhesive which can be employed for instance as a construction adhesive in the manufacture of disposable absorbent articles.

### CROSS REFERENCE TO RELATED APPLICATIONS

This application claims the benefit of U.S. Provisional Application No. 61/540,879, filed Sep. 29, 2011.

### FIELD OF THE INVENTION

The invention generally relates to stabilized alkyd resin-containing adhesives and use thereof. More specifically, the disclosure relates to construction adhesives predominantly containing oil-modified alkyd resins, absorbent articles containing these adhesives, and a method of making the articles.

### BACKGROUND OF THE INVENTION

Adhesives are commonly used in the manufacture of disposable absorbent articles such as, for example, infant and adult incontinence articles and diapers. These articles are generally manufactured by combining several components and, more specifically, by employing adhesives to form an adhesive bond between different components of the article, thereby joining those components together. The components generally include a liquid-permeable topsheet, a liquid-impermeable backsheet attached to the topsheet, and an absorbent core positioned between the topsheet and the backsheet. Thus, for example, adhesives have been used to bond the topsheet and the backsheet together. These adhesives are often referred to as construction adhesives because they are used to help construct the absorbent article from its individual components. In many instances, a hot-melt adhesive is used as a construction adhesive. Adhesives also have been used to bond discrete components, such as fasteners and leg elastics or cuffs, to each other or to the article.

“Hot-melt adhesive” refers to a thermoplastic polymer composition that is heated to obtain a liquid of flowable viscosity, and after application to an adherend, cooled to obtain a solid. An adhesive bond is formed when the adhesive solidifies upon cooling to a temperature below its melt temperature or below its solidification transition temperature. Hot-melt adhesives used as construction adhesives in the manufacture of disposable absorbent articles typically include several components. These components include one or more polymers to provide cohesive strength. These polymers make up a significant part of the hot-melt adhesive composition. The composition also includes components such as, for example, a resin or analogous material (sometimes called a tackifier) to provide adhesive strength. The composition also typically includes waxes, plasticizers or other materials to modify viscosity. Still further, the composition can optionally include further additives, such as antioxidants or other stabilizers. A typical hot-melt adhesive composition might contain from about 10 to about 35 weight percent (wt %) cohesive strength polymer(s); from about 30 to about 65 wt % resin or other tackifier(s); from more than zero to about 30 wt % plasticizer or other viscosity modifier; and optionally up to about 10 wt % stabilizer and other additives such as fillers.

Previous Art, like CM3591F, US 2010286642 and US 20090068416, describes the use of thermoplastic oil modified alkyd resins (both alone as well as formulated with other suitable ingredients) as excellent hot melt adhesives, that can be satisfactorily used even in difficult applications, like construction adhesives for hygienic absorbent articles, as baby and adult diapers, feminine hygiene pads and the like. Particularly in this field of hygienic absorbent articles, a fundamental requirement is that all the used hot melt adhesives show limited variations of melt viscosity when kept/aged for a prolonged time at their processing temperature.

Application CM 3591F discloses hot melt adhesives containing alkyd resins as their main ingredient. All these alkyd resins and their derived formulations, when used as hot melt adhesives in hygienic absorbent articles, must show a variation of the melt viscosity at 130° C. after 72 hours in the molten state at said temperature that is not greater than 40%, and preferably not greater than 35%, as measured according to ASTM D 4499-95. However, owing to the more and more demanding conditions in which said hot melt adhesives are used (e.g. production lines for hygienic articles with higher running speed) such above stated limits of viscosity variation during thermal aging are no longer considered sufficient and satisfactory; and many last-generation production lines for hygienic articles need, in reality, a variation in viscosity that is substantially lower (e.g. not higher than 20%).

Application CM 3591F, discloses an example (EXAMPLE 1) that is a formulated hot melt adhesive, containing 69% by weight of a thermoplastic oil-modified alkyd resin called EM 195090, available from Reichhold Inc., EM 195090 is reported to be modified by 50% b.w. of coconut oil and is believed to be based on glycerol—as its basic polyhydric alcohol—and on phthalic acid as its basic polybasic acid. The formulation of Example 1 of CM3591F, when aged in the above mentioned condition, shows an already good thermal aging stability that is close to the upper limit of the more stringent new requirements. It shows a variation of viscosity equal to 20.71%, even if it uses only 0.3% by weight of a standard phenolic antioxidant as a stabilizing ingredient for hot melt adhesives like Hostanox 245. Therefore, this formulation might be considered almost acceptable and at the limit of the most stringent criteria of viscosity stability.

However, these stabilizing systems based only on traditional antioxidants for hot melts (like the phenolic antioxidant Hostanox 245 and other similar compounds), produced e.g. by Clariant SE and other suppliers, do not always offer an adequately robust and sufficient protection to all hot melt adhesives containing alkyd resins as their main ingredient.

To understand the possible reasons, let's consider that traditional phenolic antioxidants, such as Hostanox 245 and other similar current commercial stabilizers for traditional hot melt adhesives, are typical “radical scavengers.” These are compounds that are able to block only the radical reactions that, in the molten adhesive, are triggered off by atmospheric oxygen. These traditional “radical scavengers” antioxidants are totally inactive or insufficiently active in protecting the adhesive if radical reactions triggered by atmospheric oxygen are not the only reactions and/or are not the main reactions that may occur inside the adhesive during its thermal aging.

In many cases, the thermal aging of alkyd resins and of their formulations shows unacceptably high variations in melt viscosity upon thermal aging when the protecting system is formed only by the well known phenolic antioxidants largely used for traditional hot melt adhesives (such as Hostanox 245 and the like). This suggests that the peculiar and profoundly different chemical nature of alkyd resins (compared to traditional hot melt adhesives) may result in the variations of melt viscosity during thermal aging in said systems. It's possible that these variations are mainly determined and controlled by phenomena and reactions that are totally different from radical reactions triggered by atmospheric oxygen.

To achieve a more robust and wider protection against not only radical reactions triggered by atmospheric oxygen, but also against other types of possible aging phenomena and reactions, it is necessary to find chemical protective systems for alkyd resins and their formulated adhesives that are different from traditional phenolic type antioxidants.

Therefore a need still exists for novel protecting systems for alkyd resins and their formulated adhesives.

### SUMMARY OF THE INVENTION

Disclosed herein are several families of novel protecting systems for oil-modified thermoplastic alkyd resins. The protecting systems greatly increase the stability of alkyd resins against melt viscosity variations after thermal aging. Such resins can be used as the predominant component of a construction adhesive or another adhesive for use in absorbent articles, such as an elastic adhesive or a stretch side panel adhesive. Accordingly, one aspect of the invention disclosed herein includes such adhesives. And, as explained in more detail below, these adhesives address one or more of the above-described challenges associated with conventional construction adhesives and elastic adhesives. On the basis of this finding, disclosed herein are additional aspects of the invention directed to articles that employ these alkyd resin-containing adhesives, and methods of making the articles.

An adhesive composition of the invention includes

(a) at least about 50 wt % of an oil-modified thermoplastic alkyd resin;
(b) a copolymer or blend of copolymers, each copolymer being formed from at least two monomer types of which at least one monomer type is a polar monomer and at least one monomer type is an apolar monomer, in an amount of at least about 0.01 wt %;
(c) at least about 1 wt % solid plasticizer or blend of plasticizers; and
(d) a stabilizer selected from the group consisting of alcohols, chelating agents, hydroxytoluene and its derivatives, hydroxyanisole and its derivatives, gallic acid and its esters, hydroquinones and their derivatives, and tocopherols;
all percentages being by weight of the adhesive composition.

The stabilizers of the present invention are novel protective chemical systems that are able to greatly increase the stability of alkyd resins against melt viscosity variations in thermal aging. In one embodiment, the variation of the melt viscosity at 130° C. after 72 hours in the molten state at said temperature that is not greater than 40%, preferably not greater than 35% and more preferably not greater than 20% as measured according to ASTM D 4499-95.

In a further aspect of the invention the defined adhesive composition is used for bonding to substrates and preferably at least one, more preferably, both substrates are formed of polyolefin. For instance, both substrates may be polyolefin films or one may be a polyolefin film and one may be a polyolefin non-woven. In particular, this use is valuable where the polyolefin is polyethylene or polypropylene. As a result the invention also provides a bonded article comprising first and second components and an adhesive composition applied between and joining said article components, the adhesive being as defined above. This article may be a disposable absorbent product.

An article, for example a disposable absorbent article, according to an aspect of the invention includes disposable absorbent article components, and the inventive adhesive composition applied between and joining at least two of the disposable absorbent article components together. Particular structures of absorbent articles are also defined herein.

Thus preferably the article can be a disposable diaper which has a pair of opposing end edges and a pair of longitudinal side edges, the absorbent article comprising a top sheet, a back sheet and an absorbent core disposed between the top sheet and back sheet, the absorbent core comprising particles of superabsorbent polymer, and an adhesive composition according to the invention disposed on a portion of the disposable absorbent article, the adhesive joining at least a first portion of the disposable absorbent article to a second portion of the disposable absorbent article by an adhesive bond.

Another aspect of the invention is a method of forming an adhesive bond between components of an article, such as a disposable absorbent article. The method generally includes applying to a first article component the inventive adhesive composition at a temperature of about 80° C. to about 200° C., and joining the first article component with a second article component to form an adhesive bond as the adhesive cools to room temperature.

In one aspect of the invention the adhesive is used in a fastening system in a disposable absorbent article (rather than as a construction adhesive).

Thus the invention also provides an absorbent article comprising an absorbent assembly containing a front waist region and a rear waist region and a crotch region longitudinally between and connecting said front waist region and said rear waist region, the front waist region and the rear waist region having an open configuration and a closed configuration about the waist of a wearer, the article also comprising a pre-engaged, adhesive fastening system comprising an adhesive engaged to an adherent to close the front waist region and the rear waist region in the closed configuration, wherein said adhesive fastening system includes an adhesive composition according to the invention.

Although the articles are not limited to consumer products falling within specific categories, a representative, non-limiting list of such categories includes baby care, feminine protection, incontinence care, paper products, and household care articles. A variety of articles may fall within these categories. Exemplary baby care articles include diapers, wipes, baby bibs, baby change and bed mats. Exemplary feminine protection articles include pads, tampons, interlabial products and pantyliners. Exemplary incontinence care articles include diapers, pads, and liners. Exemplary paper products include toilet tissue, paper towels, and facial tissues.

Exemplary household care articles include sweeper products, and floor cleaning articles. As described in further detail below, the articles can include a variety of personal hygiene products such as, for example, absorbent articles including diapers, sanitary napkins, tissues, towels, and wipes, and also non-hygiene products such as, for example, packaging articles and containers.

In one aspect of the invention the adhesive is used in packaging. Thus the invention also provides a container or a packaging article containing an adhesive composition according to the invention, said adhesive composition being disposed on at least one portion or surface of said container or packaging article for joining at least said portion or surface of said container or packaging article to at least a second portion of it, or wherein said adhesive is used for joining auxiliary elements to at least one portion or surface of said container or packaging article.

Additional features of the invention may become apparent to those skilled in the art from a review of the following detailed description, taken in conjunction with the examples and the appended claims.

### DETAILED DESCRIPTION OF THE INVENTION

According to the invention thermoplastic oil-modified alkyd resins can be used as the predominant component of an adhesive, useful as a construction adhesive or another adhesive in the production of absorbent articles (e.g. elastic adhesive, stretch side panel adhesive, fastening adhesive).

Disclosed and explained in further detail herein are novel families of stabilizing compounds for these alkyd resin-containing adhesives, uses for these adhesives, articles that include these adhesives, and methods of making the articles. As described in further detail below, the articles can include a variety of personal hygiene products such as, for example, absorbent articles including diapers, sanitary napkins, tissues, towels, and wipes, and also non-hygiene products such as, for example, packaging articles and containers.

During the synthesis of alkyd resins, the physical phenomena and the chemical reactions that lead to an increasing degree of polymerization of the alkyd resin, and therefore to a variation in its melt viscosity, are known to occur only at very high temperatures. Alkyd resins are synthesized mainly through condensation reactions between polybasic acids and polyhydric alcohols. As is well known in the art, alkyd resins are typically synthesized at temperatures of about 230-250° C. and above. The temperatures can approach 300° C.

Therefore, from a purely chemical standpoint, the aging at such a relatively very low temperature like 130° C. would not be expected to lead to any significant further chemical reaction inside an alkyd resin. It would not be expected to result in any significant variation in its melt viscosity from thermal aging. However, such significant variation has been observed in many cases.

Without being linked to any theory, scientific hypothesis or explanation and without supposing of being exhaustive, one could think of a certain number of physical and chemical causes that may lead to a variation in the melt viscosity of an aged alkyd resin. For example:

1) Metallic Catalysts. Small amounts of catalysts containing various metals like Lead, Tin, Calcium, Lithium, Sodium etc. are often used in the synthesis of alkyd resins. These catalysts are generally not removed or deactivated in commercial alkyd resins and are most likely still contained inside these polymers when sold to users. One could think that these metal catalysts may activate further reactions during aging at 130° C., even if this appears highly unlikely.
In fact, such metal catalysts are designed to be active in the reactions during the synthesis of alkyd resins, at temperatures not lower than 230-250° C. and above. Therefore, even if still present in traces in commercial alkyds, these metal catalysts would be expected to be completely inactive at such relatively low aging temperatures like 130° C.
2) Residual reactive groups. The main reactions in the synthesis of oil-modified alkyd resins are essentially a series of condensation reactions that occur among three families of components: i) Polybasic Acids; ii) Polyhydric Alcohols; iii) Vegetable Oils and/or Fatty Acids derived from Vegetable Oils. We note that in all alkyd resins modified by vegetable oils, ie. by natural triglycerides, the base polyhydric alcohol is glycerol, i.e. a triol, while the polybasic acid is generally phthalic acid i.e. a di-basic acid. Therefore, in a thermoplastic alkyd resin, the required linearity of the molecular structure results in a substantial number of chemical groups that are left unreacted. Theoretically, if one considers only the main polycondensation reactions between glycerol and phthalic acid, this would lead to the conclusion that in a thermoplastic alkyd resin, at least about 33.3% (one third) of the reactive hydroxyl groups of glycerol are left unreacted.
In any case, polycondensation reactions between such relatively weak reagents like glycerol and phthalic acid, or similarly between glycerol and fatty acids, are not expected to significantly occur at a temperature as low as 130° C.
3) Residual unsaturations. Alkyd resins contain various unsaturated chemical bonds, (i.e. double or even triple Carbon-Carbon bonds). These unsaturations may derive from, for example, unsaturated polybasic acids, (e.g. when maleic acid is used as the base polybasic acid). However, most frequently unsaturated bonds in alkyds derive mainly from vegetable oils or from their fatty acids. It is well known that most vegetable oils are composed of fatty acids containing double or even triple C—C bonds that may react with oxygen in the phenomenon of “turning rancid” for many natural oils and fats.

Even when the stoichiometric ratios of the various reagents should not allow the formation of “non-thermoplastic”, tri-dimensional chemical structures in an alkyd resin (gelled alkyd resin), it is known that “gelled micro-particles” may still form. Hypothetically, these formed gelled microparticles may aggregate with time leading to larger gelled particles that may vary the macroscopically observed melt viscosity of the material.

All the above illustrated possible physical and chemical phenomena are not expected to occur at a significant level at aging temperatures as relatively low as 130° C. Therefore, the observed variation in viscosity of some alkyd resins at 130° C. remains unexplained.

In spite of this, we have surprisingly discovered that the use of three families of novel stabilizing additives, used alone or in combination, is capable of achieving extraordinarily good results in decreasing the variation of melt viscosity of alkyd resins and of derived formulated hot melt adhesives. The variation of melt viscosity is measured according to the previous aging protocol, i.e. when one measures the variation of the melt viscosity at 130° C. after 72 hours in the molten state at said temperature, measured according to ASTM D 4499-95. These three families of stabilizing additives are effective with or without the presence of traditional “radical scavengers” like phenolic antioxidants (e.g., the ones sold under the Trade Marks Hostanox or Irganox).

More specifically, the novel improved protecting agents for alkyd resins and their formulations, used as hot melt adhesives, can be grouped into three functional or chemical classes:

### Alcohols

We have discovered that the addition of limited quantities of additional alcohols in the alkyd resins dramatically decreases the variation or their melt viscosity during thermal aging. Surprisingly, such alcohols are highly active even at addition levels much lower than the expected stoichiometric ratio with the “residual reactive Groups” in a thermoplastic alkyd resin.

Alcohols that can be used to stabilize alkyd resins and their Formulations against excessive melt viscosity variations, have a chain length up to C20. Preferred alcohols are polyhydric alcohols: Diols, including Ethylene Glycol, Propylene Glycol (Propan Diol) and Other Glycols; Triols, including Glycerol, tetraols, including Pentaerythritol. A particularly preferred polyhydric alcohol is Glycerol. The alcohols useful in the present invention preferably have a boiling point not lower than 130° C.

Said alcohols are preferably added at a level from 0.001 weight percent to 15 weight percent of the adhesive composition. More preferably, they are added at a level from 0.001 weight percent to 10% b.w. and most preferably from 0.001 weight percent to 5 b.w.

### Chelating Agents

It has been found that the addition of some Chelating Agents surprisingly helps to improve the stability in thermal aging of the alkyd resins and of their formulations, even at 130° C. We note that 130° C. is greatly below the temperature at which typically alkyd resins are synthesized (at 230-250° C. or greater, and may approach 300° C.) and at which the typical metal catalysts used in the synthesis of Alkyds are designed to be active. In particular it has been found that hydroxycarboxylic acids (like citric or tartaric acid) and their salts and esters and Aminocarboxylic acids (like the Ethylene-diamine-tetra-acetic acid known also as EDTA) and their salts and esters, are particularly effective in protecting Alkyds. These chelating agents are preferably added at a level from 0.001 weight percent to 1 weight percent of the adhesive composition. More preferably, they are added at a level from 0.001 weight percent to 0.5 b.w. and most preferably from 0.001 weight percent to 0.3% b.w.

### Other Stabilizers

Some other compounds have a significantly better stabilizing antioxidant action inside alkyd resins and their formulations, compared to tradition phenolic antioxidants for hot melts (such as, e.g., the ones sold under the Trademarks Hostanox or Irganox). These compounds are hydroxytoluene and its derivatives, hydroxyanisole and its derivatives, gallic acid and its esters, hydroquinones and their derivatives, and tocopherols. Some preferred compounds include:
Butylated Hydroxytoluene (BHT)
Butylated Hydroxyanisole (BHA)
Propyl Gallate
tert-Butylhydroquinone
Vitamin E

These other stabilizers are preferably added at a level from 0.001 weight percent to 10 weight percent of the adhesive composition. More preferably, they are added at a level from 0.001 weight percent to 5 b.w. and most preferably from 0.001 weight percent to 3% b.w.

### Absorbent Articles

Generally, an absorbent article (e.g., a disposable absorbent article) according to an aspect of the invention includes absorbent article components, and an adhesive composition applied between and joining at least two of the adsorbent article components together. As described in further detail below, the adhesive composition includes oil-modified alkyd resin.

Another aspect of the invention is a method of forming an adhesive bond between components of an article, such as a disposable absorbent article. The method generally includes applying to a first article component the adhesive composition that includes oil-modified alkyd resin at a temperature of about 80° C. to about 200° C., preferably up to about 150° C., in particular not more than 140° C., and joining the first article component with a second article component to form an adhesive bond as the adhesive cools to room temperature.

“Absorbent article” is used herein to refer generally to a device that absorbs and contains liquid. One class of such articles includes a device that is placed against or in proximity to a device wearer's body to absorb and contain various exudates discharged from the body. That class of absorbent articles includes items such as diapers, pull-on diapers, pant-type garments, training pants, incontinence briefs, incontinence undergarments, absorbent inserts, diaper holders and liners, feminine hygiene garments (e.g., sanitary napkins), and the like. Another class of absorbent articles includes a device that is applied to absorb, contain, or clean various liquids. That class of absorbent articles includes paper-based items such as napkins, tissues (e.g., facial and toilet tissues), towels, and wipes.

“Construction adhesive” is used herein to refer generally to an adhesive used to join one or more major components of an absorbent article to themselves or together during the manufacturing process. The adhesive bond formed by a construction adhesive is typically intended to be a permanent bond.

“Disposable” is used herein to describe absorbent articles that generally are not intended to be laundered or otherwise restored or reused as an absorbent article (i.e., articles intended to be discarded after a single use and, possibly, to be recycled, composted or otherwise discarded in an environmentally compatible manner).

“Pant-type” is used herein to refer to an article configured such that it has a waist opening and a pair of leg openings. A pant may be placed in position on the wearer by inserting the wearer's legs into the leg openings and sliding the pant into position about a wearer's lower torso. This configuration may be permanent as in the case of conventional underwear, or may be temporary as in the case of a training pant with openable seams for removal. Additionally, absorbent articles can be constructed with refastenable features allowing the article to have both a pant-like configuration and one or more configurations which are open or not pant like.

“Longitudinal” is used herein to refer to a direction running perpendicular from a waist edge to an opposing waist edge of the article and generally parallel to the maximum linear dimension of the article. Directions within 45 degrees of the longitudinal direction are considered to be “longitudinal”.

“Lateral” is used herein to refer to a direction running from a side edge to an opposing side edge of the article and generally at a right angle to the longitudinal direction. Directions within 45 degrees of the lateral direction are considered to be “lateral”.

As used herein, the terms “elastic,” “elastomer,” “elastomeric,” “elastically,” and “stretchable” generally refer to materials which are extensible by a pulling force, and which also return to substantially their original dimensions when the external pulling force is removed. More specifically, these terms refer to a material that is able to extend to a strain of at least 50% without breaking or rupturing, and is able to recover substantially to its original dimensions after the deforming force has been removed. It will be appreciated that these terms include the term “extensible” as each term is used herein.

“Garment-facing” is used herein to refer to describe a surface that is in contact with or may be in close proximity to any garment being worn.

“Body-facing” is used herein to refer to describe a surface that is in contact with the body of a wearer or in close proximity (i.e., closer to the body than a garment-facing surface) to the body of the wearer when the article is worn.

As used herein, the term “joined” encompasses configurations whereby an element is directly secured to another element by affixing the element directly to the other element, and configurations whereby an element is indirectly secured to another element by affixing the element to intermediate member(s) which in turn are affixed to the other element.

The term “fixed,” as used when referring to elements that are “fixed” to one another, means the elements are ordinarily joined or attached together by the manufacturer of the article in a manner such that the wearer or user of the article will not be able to un-join or detach the elements during the article's ordinary use, and the elements will not become un-joined or detached through the article's ordinary wear and tear. Elements that are “fixed” to one another are not intended to be separated during normal use of the article.

As used herein, the terms “refastenable,” “releasably fastenable,” and “engageable” refer to attachment of two or more elements or portions of elements together in a manner in which they can be separated and re-attached without substantial degradation of fastener performance or damage to surrounding components of the article which would impair the article's continued use. It will be appreciated that a refastenable, releasably fastenable, or engageable component need not have an infinite life span, but it is sufficient that the components attached in a refastenable, releasably fastenable, or engageable manner can be separated and re-attached successively several times over the typical use life span of the article. It will also be appreciated that the aggressiveness of actual fastening may be reduced significantly from fastening to refastening in absolute terms, but that such reduction is not “substantial degradation” of fastener performance if the resulting refastened strength is sufficient for purposes of ordinarily using the article and fastener.

“Mechanical fastener” is used herein to refer to a fastening system or mechanism relying on physical restraint, magnetic fields, or engagement of portions of the fastener for operation. Examples of mechanical fasteners are hook and loops, hooks and hooks, buttons, snaps, tab and slot, zippers, magnet(s), and tongue and groove fasteners.

“Spiral” is used herein to refer to a shape or pattern that generally resembles a helix as viewed in a two dimensional plane from a direction substantially orthogonal to the helix's axis of rotation. The construction adhesives disclosed herein can be applied via a spiral applicator to adhere components of an absorbent article to one another.

The adhesives of the invention are usually applied as a liquid, preferably of a low viscosity. The liquid form is obtained by heating the adhesive to a point at which flow occurs. In the liquid form, the adhesive is applied to the adherend(s) and wets and flows into the crevices (if any) of the adherend(s). The adhesive then undergoes a phase change to a solid by cooling in order for the joint to acquire the necessary strength to resist shearing forces.

The alkyd resin-containing adhesive includes thermoplastic oil-modified alkyd resin. The alkyd resin component (a) is present in the adhesive composition in an amount of at least about 50 wt % based on the total weight of the adhesive composition, preferably at least about 60 wt %, more preferably at least about 65 wt % based on the total weight of the adhesive composition. Levels of alkyd resin (a) may be up to about 98.99 wt % but preferably are not more than about 90 wt %, more preferably not more than about 80 wt %.

Alkyd resins can be classified either as crosslinkable or non-crosslinkable. These are often also described as reactive/drying or non-reactive/non-drying. Cross linking may take place through reaction through atmospheric oxygen by using drying oils such as linseed oil as the modifying oil or by the inclusion of chemical crosslinkers. In the invention the resins used are generally in the non-reactive/non-drying category. This means that preferably the alkyd resins change their melt viscosity, once aged for 72 hours at the temperature of 130° C., not more than 40%, and preferably not more than 35%.

We find that in the invention it is important to choose only alkyd resins which are thermoplastic. It is also generally valuable to choose alkyd resins which are non-reactive, namely those which show good thermal stability. This means those resins having a variation in melt viscosity when held at 130° C. for 72 hours lower than 35% (test method described below).

The alkyd resins used in the invention are thermoplastic. As a result, an adhesive composition containing them as predominant component may be melted to form a flowable material and then re-cooled to form an adhesive bond.

As mentioned above, the alkyd resins used in the adhesives are preferably chosen from those that have a surface energy of not more than about 45 mN/m, preferably not more than about 40 mN/m. Preferably the surface energy of the alkyd resin is not more than about 35 mN/m and more preferably not more than about 33 mN/m. Without wishing to be bound by any particular theory, it is believed that the presence of free acid groups and/or free alcohol groups on the polymeric backbone or chain of the alkyd resin can influence the resin's surface energy. The lower the number of free acid-groups and/or free alcohol-groups, the lower the surface energy. Alkyd resins can be controllably designed by those having skill in the art to have a specified number of free acid-groups and/or free alcohol-groups. For example, one way to reduce the surface energy, is to cap these groups during the resin's manufacture with capping agents known by those having ordinary skill in the art of alkyd resin manufacture. Capping agents generally include monofunctional acids, such as benzoic acid and acetic acid, or monofunctional alcohols.

The capping of these groups is not believed to detrimentally affect other properties of the resin that make the resin suitable for use as a construction adhesive.

The alkyd resins used in the invention are generally solvent-free.

An alkyd resin is the product of a reaction between a polyhydric alcohol and an acid or acid anhydride. The alkyd resins used in the invention are oil-modified alkyd resins. In the art of alkyd resins it is usual to talk of oil-modified alkyd resins even when what is used in the synthesis of a particular alkyd is the blend of fatty acids derived by the hydrolytic treatment of an oil. Thus, herein, oil-modified alkyd resins include those in which the synthesis involves oil, namely glycerol esters of fatty acids, or the fatty acids themselves, or both. The fatty acid that is used to modify the alkyd resin, either by use of the fatty acid itself or its corresponding glycerol ester, may be saturated or unsaturated. In one embodiment, it is derived from plant or vegetable oils. In this embodiment, vegetable oils are most preferred.

“Polyhydric alcohol” refers to an alcohol having two or more alcohol (i.e., hydroxyl) functional groups. Any suitable polyhydric alcohol or combination of polyhydric alcohols may be used in accordance with the invention. Non-limiting examples of suitable polyhydric alcohols include glycerol, glycol, sugar, sugar alcohol, and combinations thereof. Non-limiting examples of glycols include ethylene glycol, propylene glycol, dipropylene glycol, butylene glycol, hexane triol, dimethylol pentane, dimethylol ethane, and the like, polymers thereof, and combinations thereof. Non-limiting examples of sugars include glucose, sucrose, fructose, raffinose, maltodextrose, galactose, xylose, maltose, lactose, mannose, erythrose, pentaerythritol, dipentaerythritol and mixtures thereof. Non-limiting examples of sugar alcohols include erythritol, xylitol, malitol, mannitol, sorbitol, and mixtures thereof. In one embodiment, sugar alcohols are derived from plant or vegetable sugars or biomass carbohydrates, including polymeric forms such as starches and celluloses. In this embodiment, plant or vegetable sugars are most preferred.

In certain specific embodiments, the polyhydric alcohol includes glycerol, mannitol, sorbitol, and combinations thereof.

Generally, the polyhydric alcohol is substantially compatible with any polymeric components with which it may be intermixed during the production of the alkyd resin. As used herein, “substantially compatible” means that the polyhydric alcohol is capable of forming a visually homogeneous mixture with polymer present in the composition in which it is intermixed when the polyhydric alcohol is heated to a temperature above the softening and/or the melting temperature of the composition.

In the manufacture of suitable alkyd resins, the polyhydric alcohol can be present in a reaction mixture in an amount of, for example, from about 2 wt % to about 70 wt %, from about 5 wt % to about 50 wt %, from about 10 wt. % to 30 wt %, or from about 15 wt % to about 25%.

Suitable acids have at least one functional group selected from the group consisting of carboxylic acid, carboxylic acid anhydride, and combinations thereof. Non-limiting examples of classes of such acids include monoacids, diacids, polyacids (acids having at least three acid groups), polymers comprising at least one acid moiety, co-polymers comprising at least one acid moiety, anhydrides thereof, and mixtures thereof. More specifically, non-limiting examples of such acids include adipic acid, sebatic acid, lauric acid, stearic acid, myristic acid, palmitic acid, oleic acid, linoleic acid, sebacic acid, citric acid, oxalic acid, malonic acid, succinic acid, glutaric acid, maleic acid, fumaric acid, phthalic acid, isophthalic acid, terephthalic acid, acrylic acid, methacrylic acid, itaconic acid, glycidyl methacrylate, and combinations thereof. In one embodiment, fatty acids are derived from plant or vegetable oils. In this embodiment, vegetable oils are most preferred. Dibasic acids are preferably derived from plant or vegetable sugars or biomass carbohydrates, including polymeric forms such as starches and celluloses, via fermentation by genetically modified organisms or via chemical conversions. For example, succinic acid is derived from glucose via fermentation as described in US2012225461 and terephthalic acid is derived from biomass carbohydrates via chemical conversions as described in U.S. Pat. No. 7,385,081. Anhydrides of such acids also may be employed within the context of the present invention. Non-limiting examples of acid anhydrides include maleic anhydride, phthalic anhydride, succinic anhydride, and combinations thereof.

Polymers and co-polymers that include at least one acid moiety, and/or their anhydrides, also may be used as the acid moiety. Non-limiting examples of suitable polymers and copolymers include those having monomer units of acrylic acid, methacrylic acid, itaconic acid, glycidyl methacrylate, anhydrides thereof, and combinations thereof.

In the manufacture of suitable alkyd resins, the acid is employed in the reaction mixture in an amount of, for example, from about 0.1 wt % to about 30 wt %, from about 1 wt % to about 20 wt %, or from about 2 wt % to about 12 wt %. In some embodiments, the molar ratio of alcohol functional groups to acidic functional groups in the composition is at least about 1:1, or at least about 4:1. In some embodiments, the molar ratio of alcohol functional groups to acidic groups in the composition is from about 1:1 to about 200:1, or from about 1:1 to about 50:1.

Suitable oils for modifying the alkyd resin include triglycerides such as, for example, tristearin, triolein, tripalmitin, 1,2-dipalmitoolein, 1,3-dipalmitoolein, 1-palmito-3-stearo-2-olein, 1-palmito-2-stearo-3-olein, 2-palmito-1-stearo-3-olein, trilinolein, 1,2-dipalmitolinolein, 1-palmito-dilinolein, 1-stearo-dilinolein, 1,2-diacetopalmitin, 1,2-distearo-olein, 1,3-distearo-olein, trimyristin, trilaurin and combinations thereof.

Suitable triglycerides should be added to the reaction mixture from which the alkyd resin is formed. Additionally, or alternatively, oils and/or processed oils containing suitable triglycerides can be added to that mixture. Non-limiting examples of oils include beef tallow, castor oil, coconut oil, coconut seed oil, corn germ oil, cottonseed oil, fish oil, linseed oil, olive oil, oiticica oil, palm kernel oil, palm oil, palm seed oil, peanut oil, rapeseed oil, safflower oil, soybean oil, sperm oil, sunflowerseed oil, tall oil, tung oil, whale oil, and mixtures thereof. Unsaturated and saturated fatty acids may be suitable. Non-limiting examples of such fatty acids include capric acid, caproic acid, caprylic acid, lauric acid, lauroleic acid, linoleic acid, linolenic acid, myristic acid, myristoleic acid, oleic acid, palmitic acid, palmitoleic acid, stearic acid, and mixtures thereof. Combinations of the aforementioned oils and fatty acids also can be employed to modify the alkyd resin.

Preferred modifying oils have relatively shorter carbon chain length. Thus, preferred modifying oils have a content of at least about 10 wt % (based on weight of modifying oil/fatty acid used), preferably at least about 40 wt %, more preferably at least about 50 wt %, in particular at least about 70 wt % fatty acids or glycerides whose carbon chain length is not more than C16, preferably not more than C 14. Preferably the modifying oil has a high content (in particular at least about 50 wt %, especially at least about 70 wt %) lauric acid C12. Coconut oil is particularly preferred.

In the oil modified alkyd resins used in the invention the level of modifying glycerol ester or fatty acid can range from about 0.1 wt % to about 70 wt %. However, it is preferred to use medium-oil alkyd resins in which the content of triglyceride/fatty acid is at least 40 wt % and not more than 60 wt % of the reaction mixture, or long-oil alkyds in which the content of triglyceride fatty acid is more than 60 wt % of the reaction mixture, for the purpose of improving the adhesion of low surface tension substrates such as polyolefins. Such alkyd resins have sufficiently high tack and hydrophobicity qualities to give good adhesion on polyolefins and other low surface tension substrates. The improved hydrophobicity of the alkyd resin is believed to contribute to the ability of the adhesive to withstand degradation by water.

In some embodiments, combinations of acid and triglyceride are present in the reaction mixture for the manufacture of the alkyd resin, in total amounts in the ranges given above for levels of triglyceride/fatty acid.

The aforementioned additional components of the oil-modified alkyd resin can be combined in any manner known by those having ordinary skill in the art. For example, these additional components can be combined with the reaction mixture that forms the alkyd resin by mixing the same at a temperature of about 200° C. to about 250° C. for a time period sufficient to form the alkyd resin.

Some commercially available alkyd resins, although by no means all, meet all of these characteristics and it is important to choose one or more alkyd resins which meet the essential and, desirably, the preferred characteristics discussed herein.

Examples of suitable alkyd resins are those commercially available from Reichhold Co. Durham (NC) under the trade name EM.

A further essential component of the adhesive compositions of the invention is (b) a copolymer or blend of copolymers. Each copolymer contains at least one polar monomer type and at least one apolar monomer type. The polar monomer type is compatible with the polar parts of the alkyd resin molecule and the apolar monomer type is compatible with the polyolefin substrate.

Preferably, the apolar monomer is an olefin monomer. Particularly preferred is ethylene. In one embodiment, the ethylene is derived by the dehydration of ethanol obtained via the fermentation of plant or vegetable sugars. The ethylene content is preferably not more than about 90 wt % based on total monomer, more preferably not more than about 85 wt % and most preferably not more than about 75 wt %. Levels above these tend to reduce compatibility with the alkyd resin.

Preferably the ethylene content is at least about 10 wt % based on total comonomer content, more preferably at least about 15 wt % and in particular at least about 20 wt %. Levels below these tend to result in insufficient adhesion on polyolefin substrates.

The polar comonomer can for instance be selected from vinyl acetate, methyl acrylate, ethyl acrylate, propyl acrylate, butyl acrylate, hexyl acrylate, ethyl hexyl acrylate, glycidyl acrylate, glycidyl methacrylate, acrylic acid, methacrylic acid, maleic acid, and maleic anhydride, and mixtures of these. In one embodiment, the acrylates can be derived from plant or vegetable sugars. For example, glucose can be fermented to yield 3-hydroxypropionic acid and the 3-hydroxypropionic acid dehydrated to acrylic acid as described in US20070219521. We find that these particular monomers when used as polar comonomer are particularly useful in that they give an appropriate degree of polarity to the copolymer that it is sufficiently polar to be compatible with the alkyd resin but is not so polar that it reduces the adhesion of the final composition on hydrophobic substrates such as polyolefin.

The content of polar comonomer, especially when it is vinyl acetate, is preferably at least 28 wt %, more preferably at least 40 wt % and in particular at least 50 wt % based on total co-monomer content.

The copolymer component (b) is present in the adhesive composition in an amount of at least about 0.01 wt % based on total adhesive formulation. Preferably it is at least about 1 wt %, more preferably at least about 5 wt %. Generally the amount is not more than about 49 wt %, more preferably up to about 40 wt %, especially up to about 30 wt %. Even amounts of not more than about 20 wt % can be effective.

We find that the inclusion of the defined copolymers not only increases the adhesion of an adhesive relative to one based only on the alkyd resin but has the capability of substantially decreasing or even completely stopping the cold flow.

The rheological behaviour of such copolymers contributes to this. The value of Tan delta (ratio G″/G′, where G″ is the viscous or loss modulus and G′ is the elastic modulus) of the copolymer is preferably significantly below 1.

Preferably also the softening point of the alkyd resin is below the glass transition temperature of the copolymer (b).

An additional essential component for use in the present invention is one or more compatible solid plasticizer(s) that are not chemically linked inside the alkyd resin(s) molecule (free plasticizers) in addition to the chemically bound internal plasticizers (oils), already used for the chemical modification of the base alkyd(s). The solid plasticizer component (c) is compatible with the formulation, namely it is miscible and mutually soluble with the other components of the formulation so that they do not separate spontaneously.

The presence of these plasticizers generally enhances the adhesion of the blend of the two base components, alkyd resin (a) and copolymer (b). However, to retain the low cold-flow of the final formulation, these plasticizers are solid at room temperature (25° C.) and preferably are solid at the intended temperature of use for hygienic absorbent articles, i.e. at 37° C.

The plasticizer or blend of plasticizers useful in the present invention is solid at room temperature. It is preferable that the melting point of the used plasticizer or blend (measured e.g. by DSC) is not lower than about 30° C., preferably not lower than about 37° C. and more preferably not lower than about 45° C. More than one type of plasticizer can be used. When component (c) is a blend then it is the blend of plasticizers that should be solid at room temperature. Thus the blend may contain plasticizer materials that are liquid at room temperature provided that the overall blend of plasticizer is solid at room temperature.

The solid plasticizer or blend of plasticizers preferably comprises solid fully hydrogenated vegetable oils. Such materials are especially advantageous because, while they substantially contribute to reduce cold flow, they also further improve the adhesion of the final formulation on polyolefin films, due to their strong apolar character.

In a preferred embodiment these solid fully hydrogenated vegetable oils are blends of glyceryl esters of fatty acids with a carbon chain length from C8 to C30 and a very low residual unsaturation as expressed by a iodine value lower than 50, a characteristic that increases both their apolar character as well as their stability upon thermal aging.

These solid fully hydrogenated glyceryl esters may be mixed, in the final plasticizer used in the present invention, with free fatty acids, both solid and liquid at room temperature with a carbon chain length from C8 to C30 as well as with non-hydrogenated or partially hydrogenated vegetable oils and waxes, provided that the overall used plasticizer, deriving from these blends, has a melting point that is higher than room temperature, preferably higher than about 30° C., preferably higher than about 37° C. and more preferably higher than about 45° C.

The solid plasticizer component (c) is preferably present in the final formulation at a level between about 1 wt % and about 49.99 wt %, preferably between about 3 wt % and about 35 wt % and more preferably between about 5 wt % and about 30 wt %.

It can be desirable that up to 50 wt %, preferably up to 35 wt %, of the used solid plasticizer component is made of a wax that has a high melting point, in particular above the typical melting point range of hydrogenated vegetable oils. Such high melting point waxes are particularly effective in reducing cold flow of the composition as a whole, without reducing the adhesion properties of the composition. High melting point resins that have been found particularly useful have melting points not lower than 80° C., preferably not lower than 100° C. and more preferably not lower than 130° C.

Non limiting examples of such high melting point waxes are solid sulfonamides, such as para-toluene-sulfonamide; low molecular weight aromatic resins such as the ones available from the Eastman Co. under the trade names Kristalex, Endex, Piccolastic and Piccotex; solid sucrose esters such as sucrose benzoate and sucrose octa-acetate; and camphor.

The addition of a solid plasticizer with a particularly high softening point not only further prevents any cold-flow, but even further increases the softening temperature of the adhesive, as measured e.g. by the temperature of crossing of the two moduli G′ and G″, the so-called Rheological Softening Temperature or Tx. Most alkyd resins have very low Tx, while for the adhesive compositions of the invention it is preferred that the Tx of the adhesive is preferably not lower than 45° C.

The adhesives of the present invention may comprise up to about 10 wt. %, preferably up to about 5 wt. %, of a compatibilizer or a blend of compatibilizers. Although generally the compatibility among the basic essential components (a), (b) or (c) may be excellent and does not cause any substantial detrimental phase separation of the single components in the final adhesive even upon prolonged aging (24 hours and more) in the molten state at the preferred processing temperature of 130° C., in some cases the addition of limited amounts of said compatibilizers may be advisable because they increase even further the ageing stability of the formulation and better homogenize the adhesive.

In particular it has been found that suitable compatibilizers for the present invention are non-polymeric and polymeric compounds that have in their molecule or in their polymeric chain at least one chemical group or one monomer that has a very high polar character, so as to be highly compatible with the alkyd resin (a); and at least another chemical group or monomer that is compatible with one of the comonomers that form the copolymer (b). Moreover, it has been found that particularly effective compatibilizers are those compounds whose non-polymeric molecule or whose polymeric chain comprise at least one strongly hydrophilic group or monomer, e.g. methyl vinyl ether, ethyl vinyl ether, pyrrolidone, ethylene oxide, vinylpyrrolidone, vinyl alcohol linked with other chemical groups that are compatible with the apolar part (especially ethylene) or with the polar part of the above described copolymers (b).

Examples of such compatibilizers, that comprise strongly hydrophilic groups or monomers, are, among non-polymeric substances, N-alkyl-pyrrolidones, and polyethoxylates such as polyethoxylated vegetable oils; while, among polymeric materials, non-limiting examples of suitable compatibilizers are ethylene-vinyl alcohol copolymers, methyl-vinyl ether maleic anhydride (or maleic acid) copolymers, isobutylene-maleic anhydride (or maleic acid) copolymers, alkylated poly-vinyl-pyrrolidones, acrylic ester-vinylpyrrolidone copolymers, acrylic acid-vinylpyrrolidone copolymers, vinyl acetate-vinyl pyrrolidone copolymers, styrene-maleic anhydride (or maleic acid) copolymers, styrene-oxazoline copolymers, and styrene-ethylene oxide copolymers.

Tackifiers are normally considered essential components of most hot melt adhesives. Often they are the major component of the hot melt adhesive, even 50% or more of the whole composition. In the present invention it has surprisingly been found that the mentioned copolymers (b), once blended with the mentioned oil-modified alkyds, do not necessarily need the use of tackifiers in order to form hot melt adhesives with high adhesion on polyolefins. Therefore in the formulations of the present invention, the level of tackifiers may be substantially zero. This gives also substantial economic advantages because most often tackifiers are the most expensive components of a hot melt adhesive. Therefore in one embodiment, the adhesives of the present invention do not contain tackifiers.

It has been however found that, in some cases, the addition of suitable tackifiers, may be useful to even further enhance the good performance of the adhesives of the present invention. Particularly good and compatible tackifiers for this use, alone or as a blend, are for example aromatic tackifiers, terpene tackifiers, rosins and rosin ester tackifiers. Their content in the final formulations of the present invention can usefully go from zero up to about 40 wt %, preferably up to about 30 wt % and more preferably up to about 20 wt %.

The adhesive compositions may include one or more additional components, as may be desired for the processing and/or end use of the composition, though these components are not always required. Additional components may be present in any suitable amount. In some embodiments, additional components may be present in an amount of from about 0.01 wt % to about 35 wt % or from about 0.1 wt % to about 20 wt %, or from about 0.1 wt % to about 10 wt %, based on the total weight of the adhesive composition. Non-limiting examples of additional components include, but are not limited to, additional polymers, processing aids, and the like.

Other polymers have been found to have sufficient compatibility with the hot melt adhesives of the present invention, so that, even if added in minor amounts, they effectively contribute to the enhancement both of the adhesive and cohesive properties of the adhesives. As non limiting examples of polymers useful for this can be mentioned styrenic block copolymers, such as SIS, SBS, SEBS, SEPS; thermoplastic polyurethanes such as those available from the Lubrizol Corporation under the trade mark Estane; ether-ester block copolymers such as those available from Du Pont Co. under the trade mark Hytrel; ether-ester-amide and ether-amide block copolymers such as those available from Arkema Co, under the trade mark Pebax; sulfonated polyesters such as those available from the Eastman Co. under the trade name AQ; thermoplastic cellulose esters such as the cellulose acetate propionates and cellulose acetate butyrates available from the Eastman Co. under the trade codes CAP and CAB. Their amount in the final composition is usually not greater than 20 wt %, preferably not greater than 10 wt %, more preferably not greater than 5 wt %.

The adhesive compositions may also include oil gelling agents; antioxidants and stabilizers; mineral fillers; pigments and dyes; perfumes; surfactants. Each of these additives may be present in amounts up to about 10 wt %.

Examples of oil gelling agents include low molecular weight polyamides such as those available from the Henkel corporation under the trademark Versamid; polyoxy aluminium salts of fatty acids C12 to C22, such as those available from the Fed Chem Company under the trade name Manalox; metal salts of fatty acids C12-C22, preferably metal palmitates and metal stearates. Magnesium stearate is particularly preferred. Oil gelling agents act as effective stabilisers for the composition and alleviate any problems of oil leaching.

Examples of mineral fillers include silica, calcium carbonate, calcium sulphate, zeolites, clays and zinc oxide.

The addition of mineral fillers is one known method of attempting to reduce cold flow. However, inclusion of these materials in large quantities (for instance above about 4 or 5 wt %) has the disadvantage of impairing the processability and tack properties. One advantage of the use of the copolymers (b) is that mineral fillers are not required to be added in large quantities for the purpose of decreasing cold flow.

The alkyd resins used in the adhesive composition impart the adhesive with thermal stability sufficient for the applications in which the adhesive is herein contemplated. The temperature range in which the adhesive can be expected to be applied ranges from about 80° C. to about 200° C. For example, in specific applications, the preferred adhesive application temperature for polyethylene is in the range from about 90° C. to about 150° C., and for polypropylene it is about 120° C. to about 150° C. In particular the target application temperature is in the range about 120° C. to about 140° C., especially about 130° C. Within that range, the alkyd resins contemplated for use in the construction adhesive herein are expected to maintain their chemical composition (e.g., no pertinent reaction or degradation) and physical properties, such as rheological properties (e.g., substantially constant viscosity).

Although the application of the present invention is not limited to consumer products or articles falling within specific categories, a representative, non-limiting list of such categories includes baby care, feminine protection, incontinence care, paper products, and household care articles. A variety of product forms may fall within each of these categories. Exemplary baby care articles include diapers, wipes, baby bibs, baby change and bed mats. Exemplary feminine protection articles include pads, tampons, interlabial products, and pantyliners. Exemplary incontinence care articles include diapers, pads, and liners. Exemplary paper products include toilet tissues, paper towels, and facial tissues. Exemplary household care articles include sweeper products, and floor cleaning products.

The adhesive comparisons of the invention can be used as construction adhesives in hygienic absorbent articles, especially diapers.

The construction adhesive can be applied using a wide variety of known application methods including but not limited to slot extrusion, sprays, including spiral sprays, and beads. Specific examples include but are not limited to:

application of the construction adhesive in a spiral spray or slot coating to join a topsheet to an underlying nonwoven layer;

application of the construction adhesive via slot coating to join an acquisition layer or a distribution layer to a core cover;

application of beads of the construction adhesive located between the nonwoven material comprising the cuff and the backsheet;

application of the construction adhesive in a spiral spray or slot coating to join the topsheet to the backsheet so as to seal the longitudinal edges of the absorbent article;

application of the construction adhesive using slot coating to join a landing zone (i.e. a receiving member) to the backsheet;

application of the construction adhesive using slot coating to join the core cover to the dusting layer;

application of the construction adhesive in a spiral spray to join the core to the backsheet.

application of the construction adhesive in a spiral spray to join the nonwoven material comprising the cuff to the nonwoven material comprising the backsheet.

The adhesives of the invention may be used for bonding substrates, especially when at least one of the substrates is formed of polyolefin, especially polyolefin films such as polyolefin or polypropylene.

In methods of bonding substrates, the adhesive composition is dispensed onto a first substrate at a temperature of from about 80° C. to about 200° C. and the first substrate is then joined to a second substrate to form an adhesive bond between the substrates as the adhesive cools to room temperature.

Preferably one or both substrates are formed of polyolefin, especially polyolefin films. In particular one or both substrates are formed of polyolefin or polypropylene.

The application temperature is preferably not more than 180° C., in particular not more than 150° C., especially not more than 140° C. It is often at least 110° C., in particular at least about 120° C. Application temperature of around 130° C. is generally preferred.

The adhesive composition of the invention can be used in bonding substrates to form absorbent articles, in particular diapers. Accordingly, one aspect of the invention includes:

a disposable absorbent article, having a pair of opposing end edges and a pair of longitudinal side edges, the absorbent article comprising:

(a) a top sheet, a back sheet, and absorbent core disposed between the top sheet and back sheet, the absorbent core comprising particles of super absorbent polymer,
(b) an adhesive as defined above disposed on a portion of the disposable absorbent article, the adhesive joining at least a first portion of the disposable absorbent article to a second portion of the disposable absorbent article by an adhesive bond.

Preferably the adhesive bond has a shear hang time of at greater than about 40 seconds as measured according to the sheer hang time test described in US 2008/0228158. Preferably the adhesive bond has peel strength not lower than 1 N/inch as measured according to the peel strength test (in this case with the two substrates which are bonded). Preferably the adhesive is applied on the disposable absorbent article by using a spiral spray method and is disposed in a spiral pattern. Preferably the spiral pattern adhesive has a frequency from 1 to 10 spirals per 2.54 cm.

In particular the adhesive may join at least a portion of the top sheet, which is formed of a non-woven to at least a portion of the back sheet, which is a film, to form an end flap seal.

In preferred embodiments the adhesive is applied to the disposable absorbent article using a slot extrusion method. The adhesive may be applied in the form of beads.

The adhesive composition of the invention may also be useful in the fastening systems for absorbent articles. Accordingly, the invention also includes an absorbent article comprising:

(i) an absorbent assembly, comprising a front waist region, and a crotch region longitudinally between and connecting said front and said rear waist regions, the front waist region and the rear waist region having an open configuration and the closed configuration about the waist of a wearer;
(ii) a pre-engaged adhesive fastening system comprising an adhesive inherent engaged to an adherent to close the front waist region and the rear waist region in the closed configuration; wherein the adhesive fastening system includes an adhesive composition of the invention.

Furthermore, the adhesives of the invention are also useful in packaging, especially in the case where the packaging materials to be adhered comprise polyolefin materials such as polyethylene and/or polypropylene. However, they are also useful in packaging where the materials to be adhered are non-polyolefin materials.

The invention also provides a container or a packaging article, for instance a bottle, a case, a box, a tray, a pouch, a bag, containing to the adhesive of the present invention, where said adhesive is disposed on at least one portion or surface of said container or packaging article for joining at least said portion or surface of said container or packaging article to at least a second portion of it or is used for joining to at least one portion or surface of such container or packaging article, auxiliary elements such as labels, caps, handles, secondary containers and the like. Said container, packaging article or auxiliary elements may be made of paper, of cardboard, of plastics, of glass, of metal, of wood, of plywood, of cloth or of nonwoven fabric.

Non-limiting examples of absorbent articles according to the present invention are diapers and sanitary napkins designed to receive and contain vaginal discharges, such as menses. Disposable sanitary napkins are designed to be held adjacent to the human body through the agency of a garment, such as an undergarment or a panty or by a specially designed belt. Examples of the kinds of sanitary napkins to which the present invention is readily adapted are shown in U.S. Pat. Nos. 4,687,478 and 4,589,876.

Generally, sanitary napkins include a liquid impervious backsheet, a liquid pervious topsheet, and an absorbent core placed between the backsheet and the topsheet. The backsheet typically includes a thermoplastic polymer composition. The topsheet may include any of the topsheet materials discussed with respect to diapers. Similarly, the absorbent core may include any of the absorbent core materials discussed with respect to diapers. It will be apparent that construction adhesive described herein can be used to adhere various components of the sanitary napkin to one another. On the other hand it will be understood the present invention is not limited to any specific sanitary napkin configuration or structure.

Sanitary paper tissue products are commercially offered in formats tailored for a variety of uses including facial tissues and napkins, toilet tissues and absorbent towels (e.g., paper towels). These products often include two or more plies of tissue, the material for which is selected from conventional tissue or special kinds of tissues, such as so-called “through-air-dried tissue” or “differential-density-tissue.” The latter is often made via a patterned paper making belt and through-air-drying technology. Generally, however, these products can be made by any process known in the art, including, for example, a conventional papermaking process and a through-air-drying papermaking process. These products employ conventional adhesives that can now be replaced by an adhesive containing a thermoplastic, oil-modified, alkyd resin, as described herein.

### EXAMPLES AND COMPARATIVE EXAMPLES

The following examples are provided to illustrate the invention, but are not intended to limit the scope thereof.

### Comparative Example 1

EM 195090 is a thermoplastic, solvent free, oil-modified alkyd resin, reported to contain about 50% b.w. of coconut oil, available from Reichhold Inc, Durham (NC). The melt viscosity at 130° C. of this material, as received from the supplier, is 2170 mPa·s. The Alkyd Resin was thermally aged, keeping it at 130° C. for 72 hours according to the ASTM D 4499-95. After thermal aging, the viscosity of the material was 3590 mPa·s. Therefore, the viscosity variation of this material, as a result of thermal aging, is equal to +65.4%

### Comparative Example 2

The same alkyd resin of the previous Comparative example, EM 195090, was protected against thermal degradation in aging by adding Hostanox 245, an antioxidant for Hot Melts available from Clariant SE (Germany). The antioxidant was added at a level of 0.3% by weight. This is a typical level of addition for the use of such an antioxidant in traditional Hot Melts. The antioxidant was added into the molten Alkyd Resin and mixed for about ten minutes so to be sure of having homogeneously dispersed the Hostanox 245 into the alkyd resin. The initial melt viscosity at 130° C. of this blend was found to be 2170 mPa·s. After aging at 130° C. for 72 hours according to ASTM D 4499-95, the melt viscosity of the material was found to have increased to 3200 mPa·s, a percent variation equal to +47.5%.

This shows that traditional “radical scavengers” for Hot Melts like Hostanox 245, offer some protection against melt viscosity variations in thermal aging of Alkyd Resins, but they are not sufficient for the most stringent requirements for their use in Hygienic Absorbent articles.

### Comparative Example 3

The same antioxidant for Hot Melt adhesives, Hostanox 245, was added in the same manner as the previous example but at a level of 1.0% by weight. The initial melt viscosity at 130° C. of this blend was found to be 2170 mPa·s. After aging at 130° C. for 72 hours according to the ASTM D 4499-95, the melt viscosity of the material increased to 3070 mPa·s, a percent variation equal to +41.5%.

This shows that traditional phenolic antioxidants for Hot melts, as Hostanox 245, even when added in massive amounts (more than three times the usual and suggested levels of use), achieve only limited and insufficient additional protection.

### Example 1

1.0% by weight of Glycerol was added to the molten Alkyd Resin EM 195090 at 130° C. and the molten mass was mixed for about ten minutes so to get a fully homogeneous blend. The initial melt viscosity at 130° C. of this blend was 2150 mPa·s. After aging at 130° C. for 72 hours according to the ASTM D 4499-95, the melt viscosity of the material was 2340 mPa·s. This is a percent variation equal to +8.8%.

Therefore, the addition of Glycerol at 1.0% b.w. was able to greatly stabilize the alkyd resin against melt viscosity variations after thermal aging.

### Example 2

The same procedure of the previous example was repeated using 2.0% by weight the amount of added Glycerol. The initial melt viscosity at 130° C. of the blend was 2020 mPa·s. After aging at 130° C. for 72 hours according to the ASTM D 4499-95, the melt viscosity of the material was 1880 mPa·s. This was a percent variation equal to −6.9%.

### Example 3

The same procedure of example 1 was repeated, substituting the 1.0% of Glycerol with 1.0% of a different alcohol, Propan Diol. The initial melt viscosity at 130° C. of the blend was 2190 mPa·s. After aging at 130° C. for 72 hours according to the ASTM D 4499-95, the melt viscosity of the material was 2490 mPa·s. This was a percent variation equal to +13.7%.

### Example 4

The same procedure of example 3 was repeated, using 0.9% of Propan Diol (instead of 1.0% Propan Diol). The initial melt viscosity at 130° C. of the blend was 2190 mPa·s. After aging at 130° C. for 72 hours according to the ASTM D 4499-95, the melt viscosity of the material was 2500 mPa·s. This was a percent variation equal to +14.2%

### Example 5

A 10.0% b.w. solution of Citric Acid in Propan Diol was prepared by adding the desired amount of anhydrous Citric Acid, increasing the temperature to 70° C. while stirring for about five minutes, until the Citric Acid was completely dissolved into the Propan Diol. Then 1.0% of the Propan Diol/Citric Acid solution (0.10% b.w. Citric Acid and 0.90% b.w. Propan Diol) was added to the molten Alkyd EM 195090 at 130° C. and mixed for about ten minutes until a fully homogeneous molten mass was obtained. The initial melt viscosity at 130° C. of the blend was 2190 mPa·s. After aging at 130° C. for 72 hours according to the ASTM D 4499-95, the melt viscosity of the material was 2380 mPa·s. This was a percent variation equal to +8.7%.

Comparing this result with the results of Examples 3 and 4, it is therefore shown that Citric Acid performs an additional stabilizing action, in addition to and in synergy with the stabilizing action performed by Propan Diol.

### Example 6

Ethylene-diamine-tetra-acetic acid (EDTA) was suspended in Propan Diol at a ratio 10% b.w. EDTA and 90% b.w. Propan Diol. In this case, the EDTA is not completely soluble in Propan Diol; so the obtained liquid contains still some EDTA crystals as finely dispersed suspended solids. 1.0% b.w. of the liquid was mixed with the molten Alkyd EM 195090 at 130° C. and mixed for about ten minutes until a fully homogeneous molten mass was obtained. The final alkyd resin blend contained 0.10% b.w. EDTA and 0.90% b.w. Propan Diol. The initial melt viscosity at 130° C. of the blend was 2200 mPa·s. After aging at 130° C. for 72 hours according to the ASTM D 4499-95, the melt viscosity of the material was 2340 mPa·s. This is a percent variation equal to +6.4%, showing that EDTA is also a powerful stabilizer for Alkyd Resins during thermal aging.

### Example 7

Butylated Hydroxytoluene (BHT) was added at a level of 1% b.w. to the molten Alkyd Resin EM 195090 and blended like in Comparative Examples 2 and 3. The initial melt viscosity at 130° C. of the blend was 2170 mPa·s. After aging at 130° C. for 72 hours according to ASTM D 4499-95, the melt viscosity of the material was 2550 mPa·s. This is a percent variation equal to +17.5%, showing that BHT is a much more effective stabilizing antioxidant for Alkyd Resins than traditional antioxidants for Hot Melts like the Hostanox products and other similar ones, sold e.g. under the Trade Mark Irganox.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm” “Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.”

“While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.”

What is claimed is:

1. An adhesive composition comprising:
(a) at least about 50 wt % of an oil-modified thermoplastic alkyd resin;
(b) a copolymer or blend of copolymers, each copolymer being formed from at least two monomer types of which at least one monomer type is a polar monomer and at least one monomer type is an apolar monomer, in an amount of at least about 0.01 wt %;
(c) at least about 1 wt % solid plasticizer or blend of plasticizers; and
(d) a stabilizer selected from the group consisting of alcohols, chelating agents, hydroxytoluene and its derivatives, hydroxyanisole and its derivatives, gallic acid and its esters, hydroquinones and their derivatives, and tocopherols.

2. A composition according to claim 1 in which the oil-modified alkyd resin (a) comprises oil in an amount of about 40 wt % to about 60 wt %.

3. A composition according to claim 1 wherein the stabilizer is an alcohol.

4. A composition according to claim 3 wherein the stabilizer is an alcohol having a chain length up to C20 and a boiling point of about 130° C. or higher.

5. A composition according to claim 3 wherein the stabilizer is an alcohol selected from the group consisting of diols, triols and tetraols.

6. A composition according to claim 3 wherein the stabilizer is an alcohol selected from the group consisting of ethylene glycol, propylene glycol (propan diol), glycerol, and pentaerythritol.

7. A composition according to claim 3 wherein the stabilizer comprises from 0.001 weight percent to 15 weight percent of the adhesive composition.

8. A composition according to claim 1 wherein the stabilizer is a chelating agent.

9. A composition according to claim 8 wherein the stabilizer is a chelating agent selected from the group consisting of hydroxycarboxylic acids, their salts and esters, aminocarboxylic acids and their salts and esters.

10. A composition according to claim 8 wherein the stabilizer is a chelating agent selected from the group consisting of citric acid, tartaric acid and ethylene-diamine-tetra-acetic acid (EDTA).

11. A composition according to claim 8 wherein the stabilizer comprises from 0.001 weight percent to 1 weight percent of the adhesive composition.

12. A composition according to claim 1 wherein the stabilizer is selected from the group consisting of hydroxytoluene and its derivatives, hydroxyanisole and its derivatives, gallic acid and its esters, hydroquinones and their derivatives, and tocopherols.

13. A composition according to claim 1 wherein the stabilizer is selected from the group consisting of butylated hydroxytoluene (BHT), butylated hydroxyanisole (BHA), propyl gallate, tert-butylhydroquinone and Vitamin E.

14. A composition according to claim 12 wherein the stabilizer comprises from 0.001 weight percent to 10 weight percent of the adhesive composition.

15. A method of bonding two substrates, the method comprising:
(I) dispensing onto a first substrate an adhesive composition according to any of claims 1 to 12 at a temperature of about 80° C. to about 200° C., and
(II) joining the first substrate to a second substrate to form an adhesive bond between the substrates as the adhesive cools to room temperature.

16. A bonded article, preferably a disposable absorbent article, comprising:
(i) first and second components; and
(ii) an adhesive composition applied between and joining said article components, the adhesive being according to claim 1.

17. An article according to claim 16 which is a disposable diaper and which has a pair of opposing end edges and a pair of longitudinal side edges, the absorbent article comprising:
(a) a top sheet, a back sheet, and an absorbent core disposed between the top sheet and back sheet, the absorbent core comprising particles of super absorbing polymer;
(b) an adhesive composition according to claim 1 disposed on a portion of the disposable absorbent article, the adhesive joining at least a first portion of the disposable absorbent article to a second portion of the disposable absorbent article by an adhesive bond.

18. An absorbent article comprising:
(i) an absorbent assembly comprising a front waist region and a rear waist region and a crotch region longitudinally between and connecting said front and said rear waist regions, the front waist region and the rear waist region having an open configuration and a closed configuration about the waist of a wearer;
(ii) a pre-engaged, adhesive fastening system comprising an adhesive engaged to an adherent to close the front waist region and the rear waist region in the closed configuration;
wherein said adhesive fastening system includes an adhesive composition according to any of claim 1.

19. Use of an adhesive composition according to claim 1 in packaging for the adhesion of two packaging materials.

20. A container or a packaging article containing an adhesive composition according to claim 1, wherein said adhesive composition is disposed on at least one portion or surface of said container or packaging article for joining at least said portion or surface of said container or packaging article to at least a second portion of it or wherein said adhesive composition is used for joining auxiliary elements to at least one portion or surface of said container or packaging article.
